(19) United States
(12) Patent Application Publication
(10) Pub. No.: US 2023/0218566 A1
(43) Pub. Date: Jul. 13, 2023

(54) Composition and Method for Treating Chronic Pain

(71) Applicant: Zelira Therapeutics Operations Pty Ltd, Perth (AU)

(72) Inventors: Richard HOPKINS, Perth (AU); Meghan Gail THOMAS, Perth (AU); Oludare ODUMOSU, Perth (AU)

(21) Appl. No.: 18/001,556

(22) PCT Filed: Jun. 11, 2021
(86) PCT No.: PCT/AU2021/050602

§ 371 Date: Dec. 12, 2022

(30) Foreign Application Priority Data
Jun. 12, 2020 (AU) 2020901947

(51) Int. Cl. A61K 31/352; A61K 31/047; A61K 31/015; A61P 25/04

(52) U.S. Cl. CPC A61K 31/352; A61K 31/015; A61K 31/047; A61P 25/04

(57) ABSTRACT

The invention relates to pharmaceutical compositions comprising Δ9-tetrahydrocannabinol (THC), cannabidiol (CBD) and cannabigerol (CBG) and a terpene component, and their use in the treatment of chronic pain. The invention also relates to methods for treating chronic pain, especially chronic pain in athletes.

### FIELD

The invention relates to pharmaceutical compositions comprising Δ⁹-tetrahydrocannabinol (THC) and cannabidiol (CBD) and a terpene component, and their use in the treatment of chronic pain. The invention also relates to methods for treating chronic pain, especially chronic pain in athletes.

### BACKGROUND

The biological activity of Cannabis is well known and has led it to become a “recreational” drug. However, with the discovery of a class of cannabinoid (CB) receptors, and the relaxation of laws regulating Cannabis use - in some jurisdictions decriminalisation - there now exists the opportunity to explore the potential of Cannabis as a source of new therapeutics.

One of the early drivers for the medicinal use of Cannabis is its analgesic or antinociceptive efficacy, with medicinal Cannabis typically being prescribed to cancer patients to assist in pain management.

One group of patients that suffer non-cancer chronic pain is those with sports injuries. Sports injuries may result from an accident while participating in sport or may be due to poor training practices, improper equipment or lack of fitness. While some injuries may be acute and treated when they happen, chronic sports injuries occur as a result of prolonged, repetitive motion, such as over-use injuries, and might not be immediately obvious as pain and inflammation may occur or build up over a period of weeks. Furthermore, professional athletes may have limited choices available for managing chronic pain.

Retired professional athletes may also suffer long term chronic pain due to injuries sustained during their professional sports careers. For example, chronic back pain or joint pain.

There is a continuing need to develop new treatments for pain management. It would therefore be advantageous to provide alternative cannabinoid-based pharmaceutical compositions that may be useful in the treatment of chronic pain.

### SUMMARY

The inventors believe that treatment of patients suffering from non-cancer chronic pain with a pharmaceutical composition comprising Δ⁹-tetrahydrocannabinol (THC) and cannabidiol (CBD) and a synergistic terpene component may provide sufficient analgesia to assist in pain management strategies and may reduce inflammation.

In one aspect of the present invention, there is provided a pharmaceutical composition comprising a cannabinoid component and a terpene component, the composition comprising:
(a) a cannabinoid component comprising Δ⁹-tetrahydrocannabinol (THC) and cannabidiol (CBD) wherein the cannabinoid component comprises:
   i) ≥ 40% w/w Δ⁹-tetrahydrocannabinol (THC),
   ii) ≤ 50% w/w cannabidiol (CBD),
   iii) ≥ 0.15% w/w Cannabigerol (CBG),
   iv) ≤ 0.5% w/w Cannabinodiol (CBN); and
(b) a terpene component comprising:
   i) β-caryophyllene in an amount of ≥ 0.4% w/w of the composition,
   ii) d-limonene in an amount of ≥ 0.2% w/w of the composition, and
   iii) β-pinene in an amount of ≥ 0.15% w/w of the composition.

In another aspect, there is provided a method of treating chronic pain, comprising administering to a subject in need thereof an effective amount of a pharmaceutical composition of the invention.

In still a further aspect, there is provided use of of one or more of a pharmaceutical composition of the invention in the manufacture of a medicament for treating chronic pain.

In yet another aspect of the invention, there is provided a pharmaceutical composition of the invention for use in treating chronic pain.

### DEFINITIONS

The term “cannabinoid” as used herein relates to any compound that has been isolated from a Cannabis plant or synthetically created that has activity involving the endocannabinoid system. The term is used to describe the relevant compound itself irrespective of its source.

The term “cannabinoid combination” is used to describe the combination of cannabinoid compounds obtained from one or more Cannabis extracts or combinations of cannabinoid compounds obtained from one or more Cannabis extracts and synthetic cannabinoids or combinations of synthetic cannabinoids.

The term “terpenes” or “terpenoids” as used herein refers to a class of hydrocarbon molecules, which often provide a unique smell. Terpenes are derived from units of isoprene, which has the molecular formula C₅H₈. The basic molecular formula of terpenes are multiples of the isoprene unit, i.e. (C₅H₈)_{n,} where n is the number of linked isoprene units. Terpenoids are terpene compounds that have been further metabolised in the plant, typically through an oxidative process, and therefore usually contain at least one oxygen atom.

The term “terpene component” is used to describe a combination of terpenes and/or terpenoid compounds that may be present in a Cannabis extract or may be added to a cannabinoid composition as separate isolated compounds.

As used herein, the terms “treating”, “treatment”, “treat” and the like mean affecting a subject, patient, tissue or cell to obtain a desired pharmacological and/or physiological effect. The effect may be prophylactic in terms of completely or partially preventing, or reducing the severity of the experienced pain and/or may be therapeutic in terms of a partial or complete cure of the underlying cause of the pain.

The term “administering” refers to providing the pharmaceutical composition to a patient suffering from or at risk of the disease(s) or condition(s) to be treated or prevented.

By “effective amount” it is meant an amount sufficient that, when administered to the patient, an amount of the drug is provided to achieve an effect. In the case of a therapeutic method, this effect may be the treatment of the specified disease and/or condition or a symptom thereof. Therefore, the “effective amount” may be a “therapeutically effective amount”. By “therapeutically effective amount” it is meant an amount sufficient that when administered to the patient an amount of active ingredient is provided to treat the disease or a symptom of the disease.

As used herein and in the appended claims, the singular forms “a,” “an,” and “the” include plural reference unless the context clearly dictates otherwise. Thus, for example, a reference to “an excipient” may include a plurality of excipients, and a reference to “a subject” may be a reference to one or more subjects, and so forth.

The term “(s)” following a noun contemplates the singular or plural form, or both.

The term “and/or” can mean “and” or “or”.

Unless the context requires otherwise, all percentages referred to herein are percentages by weight of the composition.

Various features of the invention are described and/or claimed with reference to a certain value, or range of values. These values are intended to relate to the results of the various appropriate measurement techniques, and therefore should be interpreted as including a margin of error inherent in any particular measurement technique. Some of the values referred to herein are denoted by the term “about” to at least in part account for this variability. The term “about”, when used to describe a value, preferably means an amount within ±25%, ±10%, ±5%, ±1% or ±0.1% of that value.

The term “comprising” as used in this specification means “consisting at least in part of”. When interpreting statements in this specification that include that term, the features, prefaced by that term in each statement, all need to be present but other features can also be present. Related terms such as “comprise” and “comprised” are to be interpreted in the same manner.

Before describing the present invention in detail, it is to be understood that this invention is not limited to particularly exemplified pharmaceutical compositions, methods of production or treatment, which may, of course, vary. It is also to be understood that the terminology used herein is for the purpose of describing particular embodiments of the invention only, and is not intended to be limiting.

The inventions described and claimed herein have many attributes and embodiments including, but not limited to, those set forth or described or referenced in this summary section, which is not intended to be all-inclusive. The inventions described and claimed herein are not limited to or by the features or embodiments identified in this summary section, which is included for purposes of overview illustration only and not limitation.

All publications, patents and patent applications cited herein, whether supra or infra, are hereby incorporated by reference in their entirety. However, publications mentioned herein are cited for the purpose of describing and disclosing the protocols and reagents which are reported in the publications and which might be used in connection with the invention. Nothing herein is to be construed as an admission that the invention is not entitled to antedate such disclosure by virtue of prior invention.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

### DESCRIPTION OF EMBODIMENT(S)

The present invention provides a pharmaceutical composition comprising THC and CBD and a terpene component.

CBD is the main non-psychotropic phytocannabinoid present in the Cannabis sativa plant, in some cases constituting up to 40 percent of its extract depending on extraction technique. Both animal and human studies suggest that the pharmacokinetics and pharmacodynamics of CBD are very complex. CBD appears to operate at both CB1 and CB2 endocannabinoid receptors within the endocannabinoid system (ECS) indirectly stimulating endogenous cannabinoid signaling (anadamine) by suppressing fatty acid amide hydrolase (FAAH), the enzyme that breaks down anandamide. Importantly, this enables more anandamide to remain at the receptors, which elicits anxiolytic and antidepressant like effects. This indirect agonist property at the cannabinoid receptors may also explain its promising safety profile. Furthermore, CBD has been shown to also act on the vanilloid, adenosine and serotonin receptors explaining its broad spectrum of potential therapeutic properties in animal models and humans, including anxiolytic, antidepressant, neuroprotective, anti-inflammatory and immunomodulatory actions.

THC is the main psychotropic constituent of Cannabis, its main pharmacological effects including analgesia, muscle relaxation, antiemesis, appetite stimulation and psychoactivity. THC mimics the action of the endogenous cannabinoid receptor ligands. THC is a partial agonist of CB1 receptors, which are primarily expressed in the central nervous system, especially in areas associated with pain. It is believed that THC induces analgesia by binding presynaptic CB1 receptors, inhibiting neurons activated by pain in these areas.

There is evidence that THC and CBD used in combination, act synergistically to maximize analgesic response. CBD has been demonstrated to antagonise some undesirable effects of THC including intoxication, sedation and tachycardia, while contributing analgesic, anti-emetic, and anti-carcinogenic properties.

The pharmaceutical composition of the invention may comprise THC and CBD in ratio of THC:CBD from about 2:1 to about 1:1, such as about 1.5:1 to about 1:1 or about 1:1.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

The ratio of THC to CBD may be readily determined by methods known in the art, including High-Performance Liquid Chromatography (HPLC) and Ultra Performance Liquid Chromatography (UPLC).

References to THC and CBD (and any other natural product, including cannabinoid(s), terpene(s) and terpenoid(s)) used herein include the relevant compound and pharmaceutically acceptable salts and/or solvates (including hydrates) thereof.

The THC and CBD may be combined from purified forms of the compounds, which may be purified after combined or separate extraction from a natural source, or produced synthetically or semi-synthetically. Any means known in the art for producing CBD and/or THC is contemplated. Alternatively, the pharmaceutical composition may comprise a Cannabis extract comprising THC, CBD and a terpene component.

Cannabis plants produce a diverse array of secondary metabolites, including cannabinoids, terpenes, terpenoids, sterols, triglycerides, alkanes, squalenes, tocopherols, carotenoids and alkaloids. The mix of these secondary metabolites varies depending on several factors, including Cannabis variety, part of the Cannabis plant extracted, method of extraction, processing of the extract and season.

There are several varieties of Cannabis plant, which have been described under two distinct naming conventions. One of these conventions identifies three distinct species of Cannabis plant, namely Cannabis sativa Linnaeus, Cannabis indica LAM., and Cannabis ruderalis. Another convention identifies all Cannabis plants as belonging to the Cannabis sativa L. species, with the various varieties divided amongst several subspecies, including: Cannabis sativa ssp. sativa and ssp. indica. As used herein, the term “Cannabis” refers to any and all of these plant varieties.

Extracts of Cannabis may be prepared by any means known in the art. The extracts may be formed from any part of the Cannabis plant containing cannabinoid and terpene and/or terpenoid compounds. Extracts may be formed from a leaf, seed, trichome, flower, keif, shake, bud, stem or a combination thereof. The part of the Cannabis plant may be used fresh or dried prior to extraction. All known means of drying the plant material are contemplated. In some embodiments, the extract is formed by contacting any part of the Cannabis plant with an extractant. Any suitable extractant known in the art may be used, including, for example, alcohols (e.g. methanol, ethanol, propanol, butanol, propylene glycol etc.), water, hydrocarbons (e.g. butane, hexane, etc.), oils (e.g. olive oil, vegetable oil, essential oil, etc.), a polar organic solvent (e.g. ethyl acetate, polyethylene glycol, etc.) or a supercritical fluid (e.g. liquid CO₂). The extractant may be completely or partially removed prior to incorporation of the Cannabis extract into the pharmaceutical composition, or it may be included in the pharmaceutical composition as a carrier. The extractant may be removed by heating the extract optionally under reduced pressure (e.g. under vacuum). It will be appreciated that some of the more volatile plant metabolites (such as terpenes) may also be removed with the extractant. Accordingly, in some embodiments, removing the extractant may enrich the cannabinoid fraction of the extract. In some embodiments, the extract is filtered to remove particulate material, for example, by passing the extract through filter paper or a fine sieve (e.g. a sieve with pore sizes of 5 µm).

In some embodiments, the Cannabis extract is formed by applying heat and/or pressure to the plant material. Typically, in these embodiments, no extractant is required.

In some embodiments, the extract may be obtained from a plant selected to give a specific cannabinoid profiled, for example, high THC or high CBD or a balanced THC/CBD profile. A balance THC/CBD profile is one having a ratio of 1.5:1 to 1:1.5, especially 1:1. In some embodiments the balanced THC/CBD profile includes the CBG and is therefore a balanced THC/CBD and CBG profile, where the CBD component comprises the CBG.

In some embodiments, the extraction process is chosen to remove a substantial proportion of the terpenes present so that selected terpenes in known amounts may be formulated with the cannabinoid extract to form a pharmaceutical composition with a known terpene profile.

In some embodiments, one or more additional compounds (e.g. cannabinoid, terpene or terpenoid compounds) may be added to the Cannabis extract to form the pharmaceutical composition. The addition of compounds may be to compensate for natural variations in the relative amounts of certain compounds being expressed in the Cannabis plant or may be to enhance the activity of one or more cannabinoid, terpene or terpenoid compounds present in the extract or to provide the desired amount of the compound that is added. The added compounds may be synthetic versions of the desired compounds, they may be purified compounds obtained from other Cannabis extracts, they may be terpenes obtained synthetically or from other plant sources or they may be added by blending two, more Cannabis extracts, or a combination of thereof.

The cannabinoid fraction typically accounts for the majority of the compounds present in the Cannabis extract.

In some embodiments, the Cannabis extract may comprise about 35% to about 95% by weight cannabinoids, for example, about 40% to about 90%, about 45% to about 70% or about 45% to about 55% by weight of the Cannabis extract. In some embodiments, the Cannabis extract comprises about 5% to about 65% by weight of non-cannabinoids, for example, about 5% to about 50%, about 10% to about 40% by weight or about 15% to about 30% by weight non-cannabinoids.

In some embodiments, the Cannabis extract used in the pharmaceutical composition is further purified to increase the concentration and purity of the cannabinoids in the extract. In particular embodiments, an extract is obtained from a Cannabis plant having a high THC component and an extract is obtained from a Cannabis plant having a high CBD component and the two extracts are combined to provide the cannabinoid component of the pharmaceutical composition. In some embodiments, the extract having a high THC component has a THC content of at least 80%, especially at least 85% and more especially at least 90%. In some embodiments, the extract having the high CBD component has a CBD content of at least 85%, especially at least 90%, more especially at least 95% and most especially at least 99%.

Typically, the Cannabis extract may also comprise other cannabinoids in addition to THC and/or CBD, such as any of the cannabinoids previously identified in Cannabis extracts. To date, over 100 cannabinoids have been identified in Cannabis plants. A comprehensive list of these cannabinoids may be found in Mahmoud A. El Sohly and Waseem Gul, “Constituents of Cannabis Sativa.” In Handbook of Cannabis Roger Pertwee (Ed.) Oxford University Press (2014) (ISBN: 9780199662685). Cannabinoids that have been identified in Cannabis plants include: Cannabigerol (E)-CBG-C5 (CBG), Cannabigerol monomethyl ether (E)-CBGM-C5 A, Cannabigerolic acid A (Z)-CBGA-C5 A, Cannabigerovarin (E)-CBGV-C3, Cannabigerolic acid A (E)-CBGA-C5 A, Cannabigerolic acid A monomethyl ether (E)CBGAM-C5 A and Cannabigerovarinic acid A (E)-CBGVAC3A; (±)-Cannabichromene CBC-C5, (±)-Cannabichromenic acid A CBCA-C5 A, (±)-Cannabivarichromene, (±)-Cannabichromevarin CBCV-C3, (±)-Cannabichromevarinic acid A CBCVA-C3 A; (-)-Cannabidiol CBD-C5 (CBD), Cannabidiol momomethyl ether CBDMC5, Cannabidiol-C4 CBD-C4, (-)-Cannabidivarin CBDV-C3, Cannabidiorcol CBD-C1, Cannabidiolic acid CBDA-C5, Cannabidivarinic acid CBDVA-C3; Cannabinodiol CBND-C5, Cannabinodivarin CBND-C3; Δ⁹-Tetrahydrocannabinol Δ⁹-THC-C5 (THC), Δ⁹-Tetrahydrocannabinol-C4 Δ⁹-THCC4 (also known as tetrahydrocannabutol THCB), Δ⁹-Tetrahydrocannabivarin Δ⁹-THCV-C3, Δ⁹-Tetrahydrocannabiorcol Δ⁹-THCO-C1, Δ⁹-Tetrahydrocannabinolic acid A Δ⁹-THCA-C5 A, Δ⁹-Tetrahydrocannabinolic acid B Δ⁹-THCA-C5 B, Δ⁹-Tetrahydrocannabinolic acid-C4 A and/or B Δ⁹-THCA-C4 A and/or B, Δ⁹-Tetrahydro-cannabivarinic acid A Δ⁹-THCVA-C3 A, Δ⁹-Tetrahydrocannabiorcolic acid A and/or B Δ⁹-THCOA-C1 A and/or B), (-)-Δ⁸-trans-(6aR,10aR)-Δ⁸-Tetrahydrocannabinol Δ⁸-THC-C5, (-)-Δ⁸-trans-(6aR,10aR)-Tetrahydrocannabinolic acid A Δ⁸-THCA-C5 A, (-)-(6aS,10aR)-Δ⁹-Tetrahydrocannabinol (-)-cis-Δ⁹-THC-C5; Cannabinol CBN-C5, Cannabinol-C4 CBN-C4, Cannabivarin CBN-C3, Cannabinol C2 CBN-C2, Cannabiorcol CBN-Cl, Cannabinolic acid A CBNA-C5 A, Cannabinol methyl ether CBNM-C5, (-)-(9R,10R)-trans-Cannabitriol (-)-trans-CBT-C5, (+)-(9S,10S)-Cannabitriol (+)-trans-CBT-C5, (±)-(9R,10S/9S,10R)-cis-Cannabitriol (±)-cis-CBT-C5, (-)-(9R,10R)-trans-10-O-Ethyl-cannabitriol (-)-trans-CBT-OEt-C5, (±)-(9R, 1 0R/9S, 1 0S)-Cannabitriol-C3 (±)-trans-CBT-C3, 8,9-Dihydroxy-Δ^{6a(10a)}-tetrahydrocannabinol 8,9-Di-OH-CBT-C5, Cannabidiolic acid A cannabitriol ester CBDA-C5 9-OH-CBT-C5 ester, (-)-(6a,9S,10S,10aR)-9,10-Dihydroxyhexahydrocannabinol, Cannabiripsol, Cannabiripsol-C5, (-)-6a,7,10a-Trihydroxy-Δ⁹-tetrahydrocannabinol (-)-Cannabitetrol, 10-Oxo-Δ^{6a(10a)}tetrahydrocannabinol OTHC); (5aS,6S,9R,9aR)-Cannabielsoin CBE-C5, (5aS,6S,9R,9aR)-C3-Cannabielsoin CBE-C3, (5aS,6S,9R,9aR)-Cannabielsoic acid A CBEA-C5 A, (5aS,6S,9R,9aR)-Cannabielsoic acid B CBEA-C5 B; (5aS,6S,9R,9aR)-C3-Cannabielsoic acid B CBEA-C3 B, Cannabiglendol-C3 OH-iso-HHCV-C3, Dehydrocannabifuran DCBF-C5, Cannabifuran CBF-C5), (-)-Δ⁷-trans-(1R,3R,6R)-Isotetrahydrocannabinol, (±)-Δ⁷-1,2-cis-(1R,3R,6S/1 S,3S,6R)-Isotetrahydrocannabivarin, (-)-Δ⁷-trans-(1R,3R,6R)-lsotetrahydrocannabivarin; (±)-(laS,3aR,8bR,8cR)-Cannabicyclol CBL-C5, (±)-(1aS,3aR,8bR,8cR)-Cannabicyclolic acid A CBLA-C5 A, (±)-(laS,3aR,8bR,8cR)-Cannabicyclovarin CBLV-C3; Cannabicitran CBTC5; Cannabichromanone CBCN-C5, CannabichromanoneC3 CBCN-C3, and Cannabicoumaronone CBCON-C5.

The cannabinoid component of the pharmaceutical composition of the present invention comprises:
i) ≥ 40% w/w Δ⁹-tetrahydrocannabinol (THC),
ii) ≤ 50% w/w cannabidiol (CBD),
iii) ≥ 0.15% w/w Cannabigerol (CBG),
iv) ≤ 0.5% w/w Cannabinodiol (CBN).

In particular embodiments, the THC is present in an amount of about 40% w/w to about 75% w/w of the cannabinoid component, especially about 50% w/w to 60% w/w and more especially about 50% w/w to 55% w/w and most especially about 50% w/w. In particular embodiments, the combination of CBD and CBG together make up the remaining about 25% w/w to 50% w/w of the cannabinoid component, especially 40% w/w to 50% w/w, more especially 45% w/w to 50% w/w and most especially about 50% w/w, with other cannabinoids present in negligible amounts. In some embodiments, the CBD and CBG are present in a ratio of 1: 0.001 to 0.05, especially 1: 0.001 to 0.01, more especially 1: 0.001 to 0.005, most especially about 1: 0.003.

In some embodiments, the cannabinoid component may comprise cannabichromene (CBC) in an amount of about 0.001% w/w to about 10% of the cannabinoid component, especially about 0.001% w/w to 5% w/w of the cannabinoid fraction.

The amount of cannabinoid component in the pharmaceutical composition is in the range of about 1% to about 10% w/w, especially about 1% to about 8% w/w, more especially about 1% to about 5% w/w and most especially about 2.5 to 3.5% w/w. For example, in some embodiments, the cannabinoid component is present in the pharmaceutical composition in an amount of about 3.1% w/w of the composition.

In some embodiments, certain cannabinoids may be absent, or present in non-detectable amounts (e.g. less than about 0.001% by weight of the analyte). In some embodiments, the Cannabis extract may exclude one or more of the following cannabinoids: Δ⁹-Tetrahydrocannabinolic acid (THCA), Δ⁹-Tetrahydrocannabivarin (THCV), Cannabidiolic acid (CBDA), Cannabinodiol (CBN), (-)-Cannabidivarin (CBDV) and Cannabichromene (CBC). In particular embodiments, the pharmaceutical composition contains undetectable amounts of CBN. CBN is a degradation product of THC and care may be taken to reduce CBN formation during preparation by using low temperatures such as below 25° C. and protecting the extracts from light. Similarly, after preparation, compositions may be protected from light and maintained at lower temperatures such as below 25° C. Without being bound by theory, it is thought that the combination of terpenes present in the formulation may also stabilize the THC from degradation.

The pharmaceutical composition also comprises a terpene component. The terpene component is present in an amount in the range of about 0.75% w/w to about 10% w/w of the composition, especially about 0.75% to 5% w/w, more especially about 0.75% to about 2% w/w and more especially about 0.75% w/w to about 1.25 % w/w of the composition.

The efficacy of a composition may be enhanced when the terpene component has a certain profile, i.e. a certain proportion of particular terpenes/terpenoids are present in the composition. It is believed that the increase in efficacy may be synergistic (i.e. non-additive). It is also believed that the presence of specific components in the terpene fraction may enhance the patient’s tolerance to cannabinoid therapy.

The pharmaceutical composition of the present invention comprises:
i) β-caryophyllene in an amount of ≥ 0.4% w/w of the composition,
ii) d-limonene in an amount of ≥ 0.2% w/w of the composition, and
iii) β-pinene in an amount of ≥ 0.15% w/w of the composition.

In some embodiments, the β-caryophyllene is present in an amount of from about 0.4 % to about 5% w/w of the composition, especially about 0.4% w/w to about 2% w/w of the composition, more especially about 0.4% w/w to about 1.0% w/w of the composition, especially about 0.4% w/w to about 0.5% w/w of the composition.

In some embodiments, the d-limonene is present in an amount of from about 0.2% to about 5% w/w of the composition, especially about 0.2% w/w to about 2% w/w of the composition, more especially about 0.2% w/w to about 1.0% w/w of the composition, especially about 0.2% w/w to about 0.3% w/w of the composition.

In some embodiments, the β-pinene is present in an amount of from about 0.15% to about 5% w/w of the composition, especially about 0.15% w/w to about 2% w/w of the composition, more especially about 0.15% w/w to about 1.0% w/w of the composition, especially about 0.15% w/w to about 0.25% w/w of the composition.

The terpene component may be made up of terpenes added to the composition and terpenes that are present in the Cannabis extract from which the cannabinoid component is prepared.

A variety of terpenes and terpenoids have been identified in Cannabis extracts, including monoterpenes, monoterpenoids, sesquiterpenes and sesquiterpenoids. For example, the following terpenes and terpenoids have been identified in Cannabis extracts: Alloaromadendrene, allyl hexanoate, benzaldehyde, (Z)-a-cis-bergamotene, (Z)-a-trans-bergamotene, ß-bisabolol, epi-α-bisabolol, ß-bisabolene, borneol (camphol), cis- γ -bisabolene, bomeol acetate (bomyl acetate), α-cadinene, camphene, camphor, cis-carveol, caryophyllene (ß-caryophyllene), α-humulene (α-caryophyllene), γ-cadinene, Δ-3-carene, caryophyllene oxide, 1,8-cineole, citral A, citral B, cinnameldehyde, α-copaene (aglaiene), γ-curcumene, ß-cymene, p-cymene, β-elemene, γ-elemene, ethyl decadienoate, ethyl maltol, ethyl propionate, ethylvanillin, eucalyptol, α-eudesmol, ß-eudesmol, γ-eudesmol, eugenol, cis-ß-farnesene ((Z)-ß-farnesene), trans-α-farnesene, trans-β-farnesene, trans-γ-bisabolene, fenchone, fenchol (norbomanol, ß-fenchol), geraniol, α-guaiene, guaiol, gurjunene, methyl anthranilate, methyl salicylate, 2-methyl-4-heptanone, 3-methyl-4-heptanone, hexyl acetate, ipsdienol, isoamyl acetate, lemenol, limonene, d-limonene (limonene), linolool (linalyl alcohol, ß-linolool), α-longipinene, menthol, γ-muurolene, myrcene (ß-myrcene), nerolidol, trans-nerolidol, nerol, ß-ocimene (cis-ocimene), octyl acetate, α-phellandrene, phytol, α-pinene (2-pinene), ß-pinene, pulegone, sabinene, cis-sabinene hydrate (cis-thujanol), ß-selinene, α-selinene, γ-terpinene, terpinolene (isoterpine), terpineol (α-terpineol), terpineol-4-ol, α-terpinene (terpilene), α-thujene (origanene), vanillin, viridiflorene (ledene), and α-ylange. In some embodiments, the pharmaceutical composition comprises one or more of these terpenes and/or terpenoids, for example, the terpene fraction may comprise 2, 3, 4, 5, 6, 7, 8, 9, 10 or more of these compounds. In some embodiments, the terpene fraction comprises all of the above terpene and terpenoid compounds.

In some embodiments, the terpene component comprises only ß-caryophyllene, d-limonene and ß-pinene. In some embodiments, the terpene component may comprise one or more other terpenes derived from the Cannabis extract(s) from which the cannabinoid fraction was derived. These other terpenes may be any terpene other than β-caryophyllene, d-limonene and ß-pinene, found in Cannabis extracts and may be present in the pharmaceutical composition in a total amount in the range of 0.001% to 5% by weight of the terpene fraction.

In some embodiments, the terpene component may comprise one or more additional terpenes selected from ß-myrcene, α-terpinene, linalool, α-phellandrene, camphene, terpinolene, p-cymene, 1,8-cineole, α-bisabolol, γ-terpinene, α-pinene and guaiol. For example, the terpene component may comprise one, two, three, four, five or more of these terpenes/terpenoids. Each of these terpenoids may be absent or may be present in an amount in the range of 0.001% to 10% by weight of the terpene fraction.

In some embodiments, the terpene fraction comprises at least one of ß-myrcene, α-terpinene, linalool, α-phellandrene, camphene, terpinolene, p-cymene, 1,8-cineole, γ-terpinene and α-pinene especially at least two, at least three or at least four of these terpene/terpenoids.

In some embodiments, the terpene fraction comprises at least one of ß-myrcene, α-terpinene, linalool and α-phellandrene, especially two, three or four of these terpenes. In some embodiments the terpene fraction comprises all of ß-myrcene, α-terpinene, linalool and α-phellandrene.

In some embodiments, the terpene fraction comprises at least one of the combinations ß-myrcene and α-terpinene; ß-myrcene and linalool; ß-myrcene and α-phellandrene; α-terpinene and linalool; α-terpinene and α-phehandrene; linalool and α-phellandrene; ß-myrcene, α-terpinene and linalool; ß-myrcene, α-terpinene and α-phehandrene; ß-myrcene, linalool and α-phehandrene; α-terpinene, linalool and α-phehandrene; and ß-myrcene, α-terpinene, linalool and α-phellandrene or any of the above combinations with one or more terpene/terpenoids selected from camphene, terpinolene, p-cymene, 1,8-cineole and ß-caryophyllene.

In some embodiments, specific terpenes or terpenoids may be absent, or present in non-detectable amounts (e.g. less than about 0.001% by weight of the terpene component).

The identity and amounts of terpenes and/or terpenoids in a Cannabis extract may be determined by methods known in the art, including gas chromatography (GC). Typically, the profile of a cannabinoid fraction and a terpene fraction of a Cannabis extract are determined separately using different analytical techniques.

The pharmaceutical composition comprises a cannabinoid component and a terpene component. In some embodiments, the pharmaceutical composition comprises a cannabinoid component, a terpene component and optionally one or more pharmaceutically acceptable excipients, such as a carrier.

In some embodiments, the pharmaceutical composition comprises a cannabinoid component comprises THC and CBD obtained from one or more extracts of Cannabis plants. In some embodiments, the cannabinoid component is supplemented with one or more of THC and CBD, to provide the required ratios. In some embodiments, the terpene component is derived from a Cannabis extract. However, in particular embodiments, the terpene component in the pharmaceutical composition is provided by adding β-caryophyllene, d-limonene and ß-pinene to the composition in the required amounts thereby providing a standardized composition. The addition of compounds may be to compensate for natural variations in the relative amounts of certain compounds being expressed in the Cannabis plant or may be to enhance the activity of one or more cannabinoid, terpene or terpenoid compounds present in an extract or to provide the desired amount of the compound that is added. Terpenes may also assist in increasing absorption of the cannabinoids in the composition. Terpenes and/or terpenoids may be added to adjust their content in the pharmaceutical composition to compensate for loss during an extraction process or to provide a desired non-natural terpene/terpenoid content in the pharmaceutical composition. The added compounds may be synthetic versions of the desired compounds, they may be purified compounds obtained from other Cannabis extracts or from other plant extracts, or they may be added by blending two or more Cannabis extracts.

In some embodiments, the pharmaceutical composition optionally comprises one or more pharmaceutically acceptable excipient(s). The excipient may be a carrier, diluent, adjuvant, or other excipient, or any combination thereof, and “pharmaceutically acceptable” meaning that they are compatible with the other ingredients of the pharmaceutical composition and are not deleterious to a patient upon or following administration. The pharmaceutical compositions may be formulated, for example, by employing conventional solid or liquid vehicles or diluents, as well as pharmaceutical additives of a type appropriate to the mode of desired administration (for example, excipients, binders, preservatives, stabilisers, flavours, etc.) according to techniques such as those well known in the art of pharmaceutical formulation (See, for example, Remington: The Science and Practice of Pharmacy, 21st Ed., 2005, Lippincott Williams & Wilkins). The pharmaceutically acceptable carrier may be any carrier included in the United States Pharmacopeia/National Formulary (USP/NF), the British Pharmacopoeia (BP), the European Pharmacopoeia (EP), or the Japanese Pharmacopoeia (JP). In some embodiments, the excipient may be non-natural (e.g. synthetically produced).

The pharmaceutical composition includes those suitable for oral, rectal, nasal, topical (including oro-mucosal such as buccal and sublingual), vaginal or parenteral (including intramuscular, sub-cutaneous and intravenous) administration or in a form suitable for administration by inhalation or insufflation. In particular embodiments, the pharmaceutical composition is formulated for oral administration.

The ingredients of the pharmaceutical composition may be placed into the form of pharmaceutical compositions and unit dosages thereof, and in such form may be employed as solids, such as tablets or filled capsules or syringes, or liquids such as solutions, suspensions, emulsions, elixirs, tinctures or capsules filled with the same, all for oral use, in the form of suppositories for rectal administration; or in the form of sterile injectable solutions for parenteral (including subcutaneous) use.

Such pharmaceutical compositions and unit dosage forms thereof may comprise conventional ingredients in conventional proportions, with or without additional active ingredient(s), and such unit dosage forms may contain any suitable effective amount of the active ingredients commensurate with the intended daily dosage range to be employed.

For preparing pharmaceutical compositions described herein, pharmaceutically acceptable carriers can be either solid or liquid. Solid form preparations include powders, tablets, pills, capsules, cachets, suppositories, and dispensable granules. A solid carrier can be one or more substances which may also act as diluents, flavouring agents, solubilisers, lubricants, suspending agents, binders, preservatives, tablet disintegrating agents, or an encapsulating material.

Suitable carriers include magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Tablets, powders, capsules, pills, cachets, and lozenges can be used as solid forms suitable for oral administration.

Liquid form preparations include solutions, dispersions, suspensions, and emulsions, for example, water or water-propylene glycol solutions or in oils such as vegetable oils. For example, parenteral injection liquid preparations can be formulated as solutions in aqueous polyethylene glycol solution. Liquid preparations are preferred for embodiments involving sublingual administration.

In some embodiments, the pharmaceutical composition is formulated for sublingual or buccal administration. Typically, a sublingual or buccal pharmaceutical composition is a liquid; however, any other suitable dosage form known in the art may be employed including aerosols, lozenges, troches, films, foams, pastes and dissolvable tablets.

Sterile liquid form pharmaceutical compositions include sterile solutions, suspensions, emulsions, syrups, tinctures and elixirs. The active ingredient(s) may be suspended in a pharmaceutically acceptable carrier, such as sterile water, sterile organic solvent or a mixture of both or an oil such as medium chain triglyceride (MCT) oil.

Other liquid form preparations include those prepared by combining the cannabinoid component and terpene component with one or more naturally derived oils (e.g. an essential oil) or waxes. An “essential oil” is an oil derived by extraction (e.g. steam extraction, or contacting the plant material with an extractant) or pressing, which contains primarily hydrophobic, and generally fragrant, components of the plant material. Suitable naturally derived oils and waxes include Sesame oil, Olive oil, Arnica essential oil, Lavender essential oil, Lavender Spike essential oil, Frankincense essential oil, Lemongrass essential oil, Cinnamon Leaf essential oil, Rosemary Cineole essential oil, Rosemary essential oil, Bergamot essential oil, Myrrh essential oil, Sage essential oil, Coconut oil, Bees wax and Hemp oil.

The pharmaceutical compositions may be formulated for parenteral administration (e. g. by injection, for example bolus injection or continuous infusion) and may be presented in unit dose form in ampoules, pre-filled syringes, small volume infusion or in multi-dose containers optionally with an added preservative. The pharmaceutical compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilisation from solution, for constitution with a suitable vehicle, e.g. sterile, pyrogen-free water, before use.

Pharmaceutical forms suitable for injectable use include sterile injectable solutions or dispersions, and sterile powders for the extemporaneous preparation of sterile injectable solutions. They should be stable under the conditions of manufacture and storage and may be preserved against oxidation and the contaminating action of microorganisms such as bacteria or fungi.

The solvent or dispersion medium for the injectable solution or dispersion may contain any of the conventional solvent or carrier systems, and may contain, for example, water, ethanol, polyol (for example, glycerol, propylene glycol and liquid polyethylene glycol, and the like), suitable mixtures thereof, and vegetable oils.

Pharmaceutical forms suitable for injectable use may be delivered by any appropriate route including intravenous, intramuscular, intracerebral, intrathecal, epidural injection or infusion.

Sterile injectable solutions are prepared by incorporating the active ingredients in the required amount in the appropriate carrier with various other ingredients such as those enumerated above, as required, followed by sterilisation. Generally, dispersions are prepared by incorporating the various sterilised active ingredients into a sterile vehicle which contains the basic dispersion medium and the required other ingredients from those enumerated above. In the case of sterile powders for the preparation of sterile injectable solutions, preferred methods of preparation are vacuum drying or freeze-drying of a previously sterile suspension of the active ingredient plus any additional desired ingredients.

For oral administration, the active ingredient(s) may be incorporated with excipients and used in the form of ingestible tablets, buccal tablets, troches, capsules, elixirs, suspensions, syrups, tinctures, wafers, and the like.

The amount of active ingredient(s) in a therapeutically useful pharmaceutical composition should be sufficient that a suitable dosage will be obtained. Accordingly, the active ingredient(s) are preferably provided in an effective amount.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, lemon or cherry flavouring. When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active ingredient(s), sucrose as a sweetening agent, methyl and propylparabens as preservatives, a dye and flavouring such as cherry, lemon or orange flavour. Of course, any material used in preparing any dosage unit form should be pharmaceutically pure and substantially non-toxic in the amounts employed. In addition, the active ingredient(s) may be incorporated into sustained-release preparations and formulations, including those that allow delivery to specific regions of the gut.

Aqueous solutions can be prepared by dissolving the active ingredient(s) in water and adding suitable colorants, flavours, stabilising and thickening agents, as desired. Aqueous suspensions can be made by dispersing the finely divided active ingredient(s) in water with viscous material, such as natural or synthetic gums, resins, methylcellulose, sodium carboxymethylcellulose, or other well-known suspending agents.

Pharmaceutically acceptable carriers and/or diluents include any and all solvents, dispersion media, coatings, antibacterial and antifungal agents, isotonic and absorption delaying agents and the like.

Also included are solid form preparations that are intended to be converted, shortly before use, to liquid form preparations for oral and/or sublingual administration. Such liquid forms include solutions, suspensions, and emulsions. These preparations may contain, in addition to the active ingredient(s), colorants, flavours, stabilisers, buffers, artificial and natural sweeteners, dispersants, thickeners, solubilising agents, and the like.

For topical administration to the epidermis the active ingredient(s) may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Formulations suitable for topical administration in the mouth (oro-mucosal e.g. sublingual or buccal administration) include any liquid formulation described herein, preferably liquid formulations with a viscosity suitable for administration by dropper or syringe; lozenges comprising active ingredient(s) in a flavoured base, usually sucrose and acacia or tragacanth; pastilles comprising the active ingredient(s) in an inert base such as gelatin and glycerin or sucrose and acacia; and mouthwashes comprising the active ingredient(s) in a suitable liquid carrier.

For administration to the nasal cavity, solutions or suspensions may be applied directly to the nasal cavity by conventional means, for example with a dropper, pipette or spray. The formulations may be provided in single or multidose form. In the latter case of a dropper or pipette, this may be achieved by the patient administering an appropriate, predetermined volume of the solution or suspension.

In the case of a spray, this may be achieved for example by means of a metering atomising spray pump. For such sprays, active ingredient(s) may be encapsulated with cyclodextrins, or formulated with other agents expected to enhance delivery and retention in the nasal mucosa.

Administration to the respiratory tract may be achieved by means of an aerosol formulation in which the active ingredient(s) are provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas.

The aerosol may conveniently also contain a surfactant. The dose of drug may be controlled by provision of a metered valve.

Alternatively, the active ingredient(s) may be provided in the form of a dry powder, for example a powder mix of the active ingredient(s) in a suitable powder base such as lactose, starch, starch derivatives such as hydroxypropylmethyl cellulose and polyvinylpyrrolidone (PVP). The pharmaceutical composition as a powder may be presented in unit dose form for example in capsules or cartridges of, e.g. gelatin, or blister packs from which the powder may be administered by means of an inhaler.

In formulations intended for administration to the respiratory tract, including intranasal formulations, the pharmaceutical composition may have a small particle size for example of the order of 5 to 10 microns or less. Such a particle size may be obtained by means known in the art, for example by micronisation.

When desired, formulations adapted to give sustained release of the active ingredient(s) may be employed.

The pharmaceutical composition may be prepared in unit dosage form. In such form, the composition is subdivided into unit doses containing appropriate quantities of the active ingredient(s). The unit dosage form can be a packaged preparation, the package containing discrete quantities of preparation, such as packeted tablets, capsules, and powders in vials or ampoules or liquids in vial or pre-packaged syringes. Also, the unit dosage form can be a capsule, tablet, cachet, or lozenge itself, or it can be the appropriate number of any of these in packaged form.

Pharmaceutical compositions for parenteral administration may also be provided in unit dosage form for ease of administration and uniformity of dosage. Unit dosage form as used herein refers to physically discrete units suited as unitary dosages for the patients to be treated; each unit containing a predetermined quantity of active material calculated to produce the desired therapeutic effect in association with the required pharmaceutical excipient. The specification for the unit dosage forms are dictated by and directly dependent on (a) the unique characteristics of the active ingredient(s) and the particular therapeutic effect to be achieved, and (b) the limitations inherent in the art of compounding such an active ingredient(s) for the treatment of living patients having a diseased condition in which bodily health is impaired.

In some embodiments, the pharmaceutical composition comprises a further active ingredient. In some embodiments, the pharmaceutical composition comprises a further active ingredient other than a cannabinoid component and terpene component. Any suitable further active ingredient may be used provided that the activity of the active ingredient, THC, CBD and the terpene terpene component is not diminished when combined. In some embodiments, the further active ingredient is an analgesic or antinoiciceptive drug. In some embodiments, the analgesic or antinoiciceptive drug is a non-opioid analgesic or antinoiciceptive drug. Suitable non-opioid analgesic or antinoiceceptive drugs include FAAH inhibitors (such as paracetamol), non-steroidal antiinflamatory drugs (NSAIDs) (such as ibuprofen, aspirin and naproxen), COX-2 inhibitors (such as refecoxib, celecoxib and etoricoxib), anti-depresants (such as amitriptyline, duloxetine, hydroxyzine, promethazine, carisoprodol, tripelennamine, clomipramine, amitriptyline), adjuvant analgesics (such as nefopam, orphenadrine, pregabalin, cyclobenzaprine, hycosine), anticonvulsants (such as carbamazepine, gabapentin), non-opioid NMDA antagonists (such as piritamide and flupiritine), stimulants (such as methylphenidate, caffeine, ephedrine, dextroamphetamine, methamphetamine, pseudoephedrine, phenylephrine and cocaine), and combinations thereof.

In some embodiments, the further active ingredient is an opioid. Suitable opioids include morphinan opioids and non-morphinan opioids, for example, oxycodone, hydrocodone, oxymorphone, morphine, codeine, fentanyl, buprenorphine, tramadol, pethidine, and combinations thereof. The pharmaceutical composition may comprise an opioid in an effective amount, or in a sub-clinical amount.

The practice of the present invention employs, unless otherwise indicated, conventional pharmaceutical, veterinary and medical techniques within the skill of the art. Such techniques are well known to the skilled worker, and are explained fully in the literature.

### Methods of Treatment

The present invention provides a method for treating chronic pain, comprising administering to a subject in need thereof an effective amount of a pharmaceutical composition of the invention.

Any pharmaceutical composition described herein may be used in this method.

Chronic pain includes any pain requiring treatment for a period of greater than 1 month, for example, 6 months, 8 months, 10 months, 1 year or longer.

In some embodiments, the chronic pain is not associated with cancer or cancer therapy (sometimes referred to as non-cancer pain).

In some embodiments, the method is for treating chronic non-cancer pain.

The methods will be understood as treating pain associated with the activity of the endocannabinoid system or the activity of any of the cannabinoid receptors, including CB1 and/or CB2.

In particular embodiments, the chronic pain may be the result of injury sustained during training or sport. In some embodiments, the subject is an athlete or a retired athlete that has chronic pain. In some embodiments, the chronic pain is a result of over use or repetitive action that develops over time. In some embodiments the chronic pain is due to degenerative action on joints and back or arthritic pain. In some embodiments, the chronic pain may be arthritic pain, back pain, joint pain, muscular pain, inflammatory pain or nerve pain.

In some embodiments, the dosage of pharmaceutical composition administered delivers cannabinoids comprising THC and CBD to the subject may be from about 1 mg to about 100 mg per day, for example, from about 1 mg to about 90 mg, about 5 mg to about 50 mg or about 5 mg to about 30 mg per day.

The effective amount of the pharmaceutical composition of the invention may be held constant throughout the dosage regimen, or it may be altered depending on the symptoms of the subject. In some embodiments, the method further comprises a step of titrating the dose of the pharmaceutical composition for an individual subject.

In some embodiments, the pharmaceutical composition may be administered 1, 2, 3, 4 or more times per day.

In some embodiments, the method is continued for a defined period, for example, one week, one month, two months and the like. The treatment may continue while the subject is having treatment for underlying cause of pain, for example, physiotherapy. In other embodiments, the method is continued long term for a period of weeks, months or years to manage long term chronic pain, particularly where the cause of pain is unknown or untreatable.

The method may also comprise administering any of the further active ingredient(s) described above including any of the opioid and non-opioid analgesic and/or antinociceptive drugs or anti-inflammatory drugs described above. This further active ingredient may be administered simultaneously, separately or consecutively with pharmaceutical compositions of the invention. By simultaneously it is meant that each of pharmaceutical composition and the other active ingredient are administered at the same time either in the same pharmaceutical composition or in separate compositions. By separately it is mean that each of pharmaceutical composition and the other active ingredient are administered at the same time in different pharmaceutical compositions and optionally by different routes of administration. By consecutively it is meant that each of pharmaceutical composition and the other active ingredient are administered separately and may be at different times. Typically, when the pharmaceutical composition and the other active ingredient are administered consecutively they are administered within 24 hours, or within 12, 8, 6, 5, 4, 3, 2, or 1 hour(s) of each other. The pharmaceutical composition may be administered before or after the other active ingredient. Further, the route of administration for the pharmaceutical composition and the other active ingredient may be the same or different.

The pharmaceutical composition may be administered by any suitable route of administration. In particular embodiments, the pharmaceutical composition is administered orally.

In still a further aspect, there is provided use of of one or more of a pharmaceutical composition of the invention in the manufacture of a medicament for treating chronic pain.

In yet another aspect of the invention, there is provided a pharmaceutical composition of the invention for use in treating chronic pain.

### EXAMPLES

The invention will be further described by way of non-limiting example(s). It will be understood to persons skilled in the art of the invention that many modifications may be made without departing from the spirit and scope of the invention.

### Example 1 – Preparation of Oral Formulation

An oral tincture was prepared from Cannabis extracts obtained by extraction of Cannabis plants with ethanol, followed by removal of extractant by heating to prepare a distillate containing a cannabinoid fraction. Two extracts were prepared, one from a Cannabis plant with high THC and one from a Cannabis plant with high CBD. The content of THC, CBD, CBG and CBN was analysed in each extract. The extracts were combined to provide an extract containing a cannabinoid component with 50% THC and 50% combined CBD and CBG. This combined composition was used to prepare the oral formulation.

An oil oral formulation was prepared by mixing the components described in Table 1

TABLE 1

Formulation

| Ingredient | Amount % by weight of composition |
|---|---|
| Cannabinoid combined composition | 3.12% |
| Medium chain triglyceride Oil (MTC) | 95.95% |
| β-pinene | 0.19% |
| Limonene | 0.22% |
| β-caryophyllene | 0.47% |
| Natural Lemon flavor | 0.05% |

The MCT oil was weighed into a Brewtech blending pot, weighing the correct mass. The blending pot was placed under an overhead stirrer. The cannabinoid composition was warmed to about 16° C. The other ingredients were weighed into a Pyrex dish. The cannabinoid composition was added to the Pyrex dish and mixed well to evenly distribute throughout the ingredients. The mixture was then added to the oil and stirred until all ingredients were fully mixed and no particulates were visible.

The formulation having 20 mg/mL of cannabinoid composition is prepared and packaged into vials having 10 mL to 120 mL volume.

### Example 2 - The Effectiveness of Medicinal Cannabis in Management of Pain in High Dose Opioid Users

A study of patients referred to a medical clinic specialising in medicinal cannabis prescriptions was undertaken. The patients consented to having their data, including medical histories and current medications, collected by the clinic. Patient data was subsequently filtered to identify sixty seven patients diagnosed with chronic non-cancer pain who were also on stable high oral morphine equivalent daily dose (oMEDD) opioid medication regiment of daily morphine equivalent dose of 60 mg to 200 mg. Analysis of the data showed that medicinal cannabis (MC) treatment resulted in a reduction in oMEDD dose, reduced the severity of pain and interference of pain symptoms in daily living, improved depression, anxiety or stress, reduced the symptoms of insomnia and to determine the optimal ratio and dose level for MC to achieve the above results.

The patients were administered a cannabis composition comprising THC and CBD in a ratio of about 1:1 at an escalating dosage over 3 visits at monthly intervals as set out in Table 2:

TABLE 2

| Cannabinoid | Median Dose (mg/kg) | | |
|---|---|---|---|
| | Visit 1 | Visit 2 | Visit 3 |
| THC | 0.104 | 0.143 | 0.157 |
| CBD | 0.152 | 0.260 | 0.233 |
| Total Cannabinoids | 0.303 | 0.48 | 0.467 |

As the cannabis composition was prepared from extracts of cannabis, the composition also comprised CBG.

The median dose of cannabinoids increased gradually, stating at 0.303 mg/kg per day, moving to 0.484 mg/kg per day at the second visit before plateauing at 0.467 mg/kg per day by the third visit. For an average adult of 70 kb, this equates to 32.7 mg of cannabinoids per day, 228.8 mg of cannabinoids per week, 915.3 mg of cannabinoids per month and 11.0 g of cannabinoids per year.

At each visit, the patient completed validated questionnaires:
Brief Pain Inventory (BPI)
Pain Severity
Pain Interference
Depression, Anxiety, Stress Scale (DASS-21)
Insomnia Severity Index (ISI).

The BPl measures two constructs of pain. The first, pain severity, refers to the individual’s self-report of the magnitude of pain that they were experiencing. This is rated on a scale from 0 (no pain) to 10 (worst pain you can imagine) and averaged across several items assessing both current pain and pain in the previous 24 hours. The second pain construct, pain interference, refers to the extent of impact that an individual’s perceived level of pain has on things such as their sleep, interactions with others and general enjoyment of life.

DASS-21 consists of 21 statements which participants are asked to consider how much each statement applied to them over the past week. The total scores for each subscale (i.e. depression, anxiety and stress) can range from 0 to 21, with the severity categories for each listed below in Table 3:

TABLE 3

| | Depression | Anxiety | Stress |
|---|---|---|---|
| Normal | 0-4 | 0-3 | 0-7 |
| Mild | 5-6 | 4-5 | 8-9 |
| Moderate | 7-10 | 6-7 | 10-12 |
| Severe | 11-13 | 8-9 | 13-16 |
| Extremely Severe | 14+ | 10+ | 17+ |

The ISI has seven questions which patients are asked to rate their current (i.e. last 2 weeks) severity. The seven answers are added to give a total score with a higher score indicating more severe insomnia:
0-7 No clinically significant insomnia
8-14 subthreshold insomnia
15-21 clinical insomnia (moderate severity)
22-28 clinical insomnia (severe)

A clinically relevant improvement in BPI, DASS and ISI was determined to have occurred if the scale score changed by 2 or more points.

In the study, of the 67 patients enrolled at baseline (time 0), 66 returned at visit 1, 48 returned for visit 2 and 32 patients returned for visit 3 at approximately 3 months from the baseline visit. Due to staggered recruitment, it is not possible to comment on drop out rates.

Over the study there was a decrease in average oMEDD from 116.63 at baseline to 87.02 mg at visit 3, a 25% reduction. The changes in individual oMEDD from the baseline assessment (Visit 0) to the first assessment following MC treatment (Visit 1) were highly variable, with some individuals increasing their oMEDD and others decreasing their oMEDD in this period. This may reflect the stepped dosing strategy associated with MC, where patients start low and go slow until their optimal MC dose is found. It should also be noted that patients were not undergoing an official opioid reduction program during the study. The changes in oMEDD are shown in Table 4:

TABLE 4

| Visit | Mean Severity | N with >2 points Change | Total N |
|---|---|---|---|
| Visit 0 | 6.43 | - | - |
| Visit 1 | 5.91 | 16 (24.2%) | 66 |
| Visit 2 | 4.97 | 12 (25.0%) | 48 |
| Visit 3 | 5.28 | 6 (18.8%) | 32 |

On average, there was a decrease in self-reported pain severity in high oMEDD non-cancer pain patients, from a mean score of 6.43 at baseline to 5.28 at Visit 3, an 18% reduction. The greatest change in the average patients’ self-reported pain severity occurred between baseline and Visit 1 after starting MC. The reduction in self-reported pain interference was clinically relevant, a reduction of 2 points or more from baseline, in 24.2% of patients at Visit 1, in 25% at Visit 2 and in 18.8% at Visit 3 as shown in Table 5.

TABLE 5

| Visit | N with >2 points Change | Total N | Mean Interference |
|---|---|---|---|
| Visit 0 | - | - | 7.05 |
| Visit 1 | 24 (36.4%) | 66 | 5.56 |
| Visit 2 | 15 (31.3%) | 48 | 5.26 |
| Visit 3 | 9 (28.1%) | 32 | 5.51 |

On average, the patterns of change in depressive severity in high oMEDD non-cancer pain patients were very similar for individuals with “Normal to Mild” depressive severity and “Moderate to Severe” depressive severity. For patients with “Moderate to Severe” depressive severity, there was a decrease from the baseline mean score of 11.05 to 10.06 at Visit 3, an 8.96% decrease. For patients with “Normal to Mild” depressive severity, there was a minor increase from the baseline mean score of 4.00 to 4.54 at Visit 3, a 13.5% increase. However, this increase was due to one highly outlying patient. If the outlying patient is removed, there was a decrease in depressive severity from a baseline mean score of 3.91 to 3.17 at Visit 3, an 18.9% decrease. The results are shown in Table 6:

TABLE 6

| Visit | Normal-Mild Severity | Moderate – Severe Severity |
|---|---|---|
| Visit 0 | 4.00 | 11.05 |
| Visit 1 | 2.92 | 8.15 |
| Visit 2 | 3.72 | 8.07 |
| Visit 3 | 4.54 | 10.6 |

A similar, but more marked pattern of change was seen with self-reported anxiety severity. For patients with “Moderate to Severe” anxiety severity, there was a decrease from the baseline mean score of 9.81 to 7.09 at Visit 3, a 27.7% decrease. On average, in the “Normal to Mild” group, there was a moderate increase in anxiety severity in the high oMEDD non-cancer pain patients from a baseline mean score of 2.69 to 3.42 at Visit 3, a 27.1% increase. However, again this is likely due to a single outlying patient. If the outlying individual is excluded, the change from baseline was 2.44 to 2.94 at Visit 3, a 20.5% increase. The results are shown in Table 7:

TABLE 7

| Visit | Normal-Mild Severity | Moderate – Severe Severity |
|---|---|---|
| Visit 0 | 2.69 | 9.81 |
| Visit 1 | 3.18 | 6.83 |
| Visit 2 | 2.68 | 6.53 |
| Visit 3 | 3.42 | 7.09 |

For patients with “Moderate to Severe” stress severity, there was a decrease from the baseline mean score of 13.54 to 10.09 at Visit 3, a 25.5% decrease. On average, in the “Normal to Mild” group, there was a moderate decrease in stress severity in the high oMEDD non-cancer pain patients from a baseline mean score of 5.56 to 5.26 at Visit 3, a 5.4% increase. The results are shown in Table 8.

TABLE 8

| Visit | Normal-Mild Severity | Moderate – Severe Severity |
|---|---|---|
| Visit 0 | 5.56 | 13.54 |
| Visit 1 | 5.16 | 9.44 |
| Visit 2 | 4.59 | 9.16 |
| Visit 3 | 5.26 | 10.09 |

For patients with “Above Threshold” insomnia severity, there was a decrease from the baseline mean score of 20.22 to 14.33 at Visit 3, a 29.1% decrease. On average, in the “Sub-Threshold” group, there was a moderate decrease in insomnia severity in high oMEDD non-cancer pain patients from a baseline mean score of 10.05 to 8.67 at Visit 3, a 13.7% decrease. The results are shown in Table 9.

TABLE 9

| Visit | Sub-Threshold | Above Threshold |
|---|---|---|
| Visit 0 | 10.05 | 20.22 |
| Visit 1 | 8.45 | 14.79 |
| Visit 2 | 8.69 | 13.09 |
| Visit 3 | 8.67 | 14.33 |

### Example 3 – Clinical Study to Evaluate the Safety, Tolerability and Pharmacokinetics of MC Formulation in Chronic Non-Cancer Pain Patients

The study was an open label dose-escalation clinical study consisting of 5 stages as follows:

Stage 1: Participants received a single dose of 2.5 mg THC/2.5 mg CBD and blood taken for pharmacokinetic (PK) analysis, after which there was a 7 day washout period.

Stage 2: Participants received a single dose of 2.5 mg THC/2.5 mg CBD following a high fat meal and blood taken for PK analysis. Participants then continued to take 2.5 mg THC/2.5 mg CBD BID (total daily dose of 5 mg THC/5mg CBD) for one week.

Stage 3: Participants received a single dose of 5 mg THC/5 mg CBD and blood taken for PK analysis. Participants then continued to take 5 mg THC/5 mg CBD BID (total daily dose of 10 mg THC/10 mg CBD) for one week.

Stage 4: Participants received a single dose of 7.5 mg THC/7.5 mg CBD and blood taken for PK analysis. Participants then continued to take 7.5 mg THC/7.5 mg CBD BID (total daily dose of 15 mg THC/15 mg CBD) for one week.

Stage 5: Participants received a single dose of 12.5 mg THC/12.5 mg CBD and blood taken for PK analysis. Participants then continued to have a 7 day washout period before returning for the close-out examination.

All doses were split and administered morning and evening and administered orally via a pre-filled syringe.

The number of participants is 9. Two participants did not complete the study.

Safety parameters monitored included number and frequency of adverse events and serious adverse events, local tolerability, vial signs including blood pressure, pulse and respiration rate, oral body temperature and physical examination. No serious adverse events occurred during the study. A number of mild adverse events were attributed to the study medication. The most common adverse event was Euphoric Mood, especially at higher levels of dosing such as stage 4. The next most common adverse event was headache, occurring at most stages during the study.

Efficacy parameters include:
BPI
DASS-21
ISI
Sleep Diary – total sleep time (sTST), time to fall asleep (sSOL), number of awakenings (sWASO), quality of sleep (sQual 5-point scale), refreshed feeling on wakening (sFRESH 5-point scale, rating of daytime energy/mood/functioning)
Self-reported opioid and other pain medication use.

The PK parameters monitored include AUCO-8h, AUC0-∞, Cmax, t½, tmax and Kel (λz).

The MC is provided in a formulation of 10 mg THC/10 mg CBD per mL as set out in Example 1. The unit dose is 0.25 mL – 1.0 mL, dose range 0.25 mL (2.5 mg) to 1.25 mL (12.5 mg).

The weekly BPI scores were obtained by averaging across the previous 7 days for each individual. The average and interquartile range, along with paired comparison t-tests were presented in Table 10 for both pain severity and interference due to pain:

TABLE 10

| Weekly Brief Pain Inventory | | Basline (Day 0) | Weekly scores | | | | | Day |
|---|---|---|---|---|---|---|---|---|
| | | | Week 1 | Week 2 | Week 3 | Week 4 | Week 5 | 36 |
| Severity | Number | 9 | 9 | 9 | 9 | 8 | 7 | 7 |
| Absolute values | Mean | 5.53 | 5.67 | 5.33 | 4.75 | 4.47 | 5.06 | 5.45 |
| | 25th Percentile | 4.75 | 4.75 | 4.43 | 4.14 | 4.33 | 4.00 | 4.31 |
| | 75th Percentile | 6.00 | 6.36 | 6.42 | 5.18 | 5.33 | 5.97 | 6.63 |
| | Minimum | 3.50 | 3.86 | 3.61 | 3.47 | 1.71 | 3.21 | 2.50 |
| | Maximum | 8.00 | 7.46 | 7.32 | 6.28 | 5.86 | 7.89 | 8.00 |
| T-test* | p-value | | ns | ns | ns (0.066) | ns (0.077) | ns | ns |
| Interference | Number | 9 | 9 | 9 | 8 | 7 | 7 | 7 |
| Absolute values | Mean | 6.19 | 5.47 | 4.95 | 4.47 | 4.05 | 4.89 | 5.17 |
| | 25th Percentile | 4.67 | 4.61 | 2.93 | 3.00 | 2.61 | 3.57 | 4.14 |
| | 75th Percentile | 7.86 | 6.67 | 5.71 | 5.56 | 5.41 | 6.22 | 6.38 |
| | Minimum | 3.57 | 2.67 | 2.78 | 1.20 | 1.14 | 2.67 | 2.43 |
| | Maximum | 9.14 | 8.67 | 7.78 | 7.30 | 7.58 | 8.00 | 1.00 |
| T-test* | p-value | | ns | 0.043 | 0.027 | 0.009 | 0.028 | ns |

*Paired t-test (comparison to baseline, two-tailed); ns =not significant at the 0.05 significance level.

These results show that there was no statistical difference in perceived severity of pain. However, there was a statistically significant improvement in interference, especially at a total daily dose of 10 mg THC/10 mg CBD (stage 3); a total daily dose of 15 mg THC/15 mg CBD (stage 4); or a single dose of 12.5 mg THC/12.5 mg CBD (Stage 5).

The DASS-21 results for depression, anxiety and stress are shown in Table11:

TABLE 11

| DASS21 | | Basline (Day 0) | Day | | | | | Day 36 |
|---|---|---|---|---|---|---|---|---|
| | | | 1 | 8 | 15 | 22 | 29 | |
| Number | | 9 | 9 | 9 | 8 | 8 | 7 | 7 |
| Depression | | | | | | | | |
| Absolute values | Mean | 10.2 | 8.7 | 7.6 | 5.4 | 4.9 | 4.4 | 4.3 |
| | Std dev | 6.7 | 5.9 | 6.4 | 5.0 | 5.0 | 3.9 | 4.1 |
| | Median | 11 | 9 | 8 | 5.5 | 4.5 | 7 | 4 |
| | Minimum | 1 | 1 | 0 | 0 | 0 | 0 | 0 |
| | Maximum | 20 | 19 | 18 | 15 | 13 | 8 | 10 |
| Change from baseline | Mean | | -1.6 | -2.7 | -4.3 | -4.8 | -5.9 | -6.0 |
| | Std dev | | 2.5 | 2.1 | 3.2 | 2.8 | 3.8 | 3.7 |
| | Median | | -2 | -3 | -4 | -5 | -5 | -5 |
| | Minimum | | -6 | -7 | -10 | -9 | -12 | -11 |
| | Maximum | | 2 | 0 | 0 | -1 | -1 | -1 |
| T-test* | | | ns | p=0.005 | p=0.007 | p=0.002 | p=0.006 | p=0.005 |
| Anxiety | | | | | | | | |
| Absolute values | Mean | 8.2 | 7.2 | 6.9 | 5.4 | 4.4 | 5.4 | 4.7 |
| | Std dev | 6.6 | 5.9 | 5.8 | 5.4 | 5.0 | 6.4 | 6.1 |
| | Median | 7 | 6 | 6 | 3 | 2 | 3 | 2 |
| | Minimum | 1 | 0 | 0 | 0 | 0 | 0 | 0 |
| | Maximum | 21 | 17 | 16 | 14 | 14 | 15 | 16 |
| Change from baseline | Mean | | -1.0 | -1.3 | -3.0 | -4.0 | -3.3 | -4.4 |
| | Std dev | | 1.4 | 1.9 | 3.0 | 2.9 | 3.7 | 5.1 |
| | Median | | -1 | -1 | -4 | -6 | -6 | -5 |
| | Minimum | | -2 | -3 | -7 | -7 | -8 | -8 |
| | Maximum | | 0 | 1 | 0 | 0 | -1 | 0 |
| T-test* | | | ns | ns | p=0.022 | p=0.007 | ns (0.054) | p=0.008 |
| Stress | Mean | 10.2 | 9.7 | 9.7 | 7.4 | 5.8 | 6.4 | 6.9 |
| Absolute values | Std dev | 6.3 | 5.7 | 6.7 | 5.7 | 4.3 | 5.9 | 5.7 |
| | Median | 11 | 11 | 8 | 6 | 6 | 8 | 7 |
| | Minimum | 1 | 0 | 0 | 0 | 0 | 0 | 0 |
| | Maximum | 20 | 18 | 20 | 16 | 12 | 15 | 17 |
| Change from baseline | Mean | | -0.56 | -0.56 | -2.25 | -3.88 | -3.86 | -3.43 |
| | Std dev | | 1.74 | 1.33 | 2.92 | 2.53 | 2.48 | 1.72 |
| | Median | | 0 | 0 | -2 | -4 | -4 | -3 |
| | Minimum | | -4 | -3 | -8 | -8 | -8 | -6 |
| | Maximum | | 2 | 1 | 2 | -1 | -1 | -1 |
| T-test* | | | ns | ns | ns (0.065) | p=0.003 | p=0.006 | p=0.002 |

*Paired t-test (comparison to baseline, two-tailed); ns = not significant at the 0.05 significance level; p = p-value.

These results show a statistically significant improvement in depression at a total daily dose of 5 mg THC/5mg CBD (stage 2), a total daily dose of 10 mg THC/10 mg CBD (stage 3); a total daily dose of 15 mg THC/15 mg CBD (stage 4); or a single dose of 12.5 mg THC/12.5 mg CBD (Stage 5), in anxiety at a total daily dose of 10 mg THC/10 mg CBD (stage 3); a total daily dose of 15 mg THC/15 mg CBD (stage 4); or a single dose of 12.5 mg THC/12.5 mg CBD (Stage 5)and in stress at a total daily dose of 15 mg THC/15 mg CBD (stage 4); or a single dose of 12.5 mg THC/12.5 mg CBD (Stage 5).

The insomnia severity index (ISI) results are shown in Tables 12:

TABLE 12

| Insomnia Severity Index | | Basline (Day 0) | Day | | | | | Day 36 |
|---|---|---|---|---|---|---|---|---|
| | | | 1 | 8 | 15 | 22 | 29 | |
| Total score | | | | | | | | |
| Number | | 9 | 9 | 8 | 8 | 7 | 7 | 7 |
| Absolute values | Mean | 17.4 | 16.4 | 16.9 | 15.8 | 8.9 | 11.7 | 13.3 |
| | Std dev | 5.7 | 3.7 | 4.5 | 3.7 | 4.0 | 7.7 | 4.2 |
| | Median | 16 | 16 | 16 | 15 | 8 | 11 | 15 |
| | Minimum | 9 | 11 | 10 | 12 | 4 | 5 | 5 |
| | Maximum | 28 | 25 | 25 | 23 | 15 | 27 | 17 |
| Change from baseline | Mean | | -1.0 | -0.7 | -1.5 | -9.5 | -5.4 | -3.9 |
| | Std dev | | 2.8 | 4.1 | 5.4 | 4.6 | 5.9 | 4.0 |
| | Median | | -1 | -1 | -1 | -9 | -5 | -3 |
| | Minimum | | -6 | -7 | -13 | -15 | -15 | -11 |
| | Maximum | | 3 | 6 | 5 | -1 | 3 | 1 |
| | T-test* | | ns | ns | ns | p=0.002 | p=0.050 | p=0.044 |

How would you rate your sleep quality in the last week?

| Number | | 9 | 9 | 9 | 6 | 8 | 7 | 7 |
|---|---|---|---|---|---|---|---|---|
| Absolute values | Mean | 3.2 | 3.1 | 3.0 | 2.3 | 2.0 | 2.0 | 2.4 |
| | Std dev | 0.8 | 0.8 | 0.7 | 0.8 | 1.1 | 1.0 | 0.5 |
| | Median | 3.0 | 3.0 | 3.0 | 2.5 | 2.0 | 2.0 | 2.0 |
| | Minimum | 2 | 2 | 2 | 1 | 1 | 0 | 2 |
| | Maximum | 4 | 4 | 4 | 3 | 4 | 3 | 3 |
| Change from baseline | Mean | | -0.11 | -0.22 | 1.00 | -1.13 | -1.14 | -0.71 |
| | Std dev | | 0.33 | 0.83 | 0.63 | 0.99 | 1.46 | 0.76 |
| | Median | | 0.0 | 0.0 | -1.0 | -1.5 | -1.0 | -1.0 |
| | Minimum | | -1 | -2 | -2 | -2 | -4 | -2 |
| | Maximum | | 0 | 1 | 0 | 0 | 0 | 0 |
| | T-test* | | ns | ns | p=0.012 | p=0.015 | ns (0.084) | p=0.047 |
| How many hours sleep have you had for the past week? | | | | | | | | |
| Number | | 9 | 8 | 6 | 6 | 7 | 6 | 4 |
| Absolute values | Mean | | 35.9 | 42.7 | 43.8 | 51.8 | 52.8 | 46.8 |
| | Std dev | 9.3 | 8.7 | 10.7 | 11.0 | 12.3 | 13.6 | 9.9 |
| | Median | 40.0 | 37.5 | 42.0 | 42.3 | 49.0 | 49.5 | 45.0 |
| | Minimum | 25 | 18 | 28 | 28 | 35 | 35 | 37 |
| | Maximum | 54 | 45 | 56 | 61 | 69 | 72 | 60 |
| Change from baseline | Mean | | -5.0 | 0.0 | 0.6 | 8.8 | 10.1 | 6.8 |
| | Std dev | | 12.8 | 7.9 | 10.4 | 13.1 | 14.3 | 9.7 |
| | Median | | 0.0 | -3.0 | -2.8 | 5.0 | 5.0 | 5.0 |
| | Minimum | | -36 | -7 | -7 | -5 | -5 | -3 |
| | Maximum | | 4 | 14 | 21 | 28 | 30 | 20 |
| T-test* | | | ns | ns | ns | ns | ns | ns |

*Paired t-test (comparison to baseline, two-tailed); ns = not significant at the 0.05 significance level; p = p-value.

These results show a perceived improvement in insomnia severity at a total daily dose of 15 mg THC/15mg CBD (stage 4); or a single dose of 12.5 mg THC/12.5 mg CBD (Stage 5) and an improvement sleep quality at a total daily dose of 10 mg THC/10 mg CBD (stage 3); a total daily dose of 15 mg THC/15 mg CBD (stage 4); or a single dose of 12.5 mg THC/12.5 mg CBD (Stage 5) but no improvement in the number of hours of sleep was observed.

Sleep diary data was averaged across the week for each participant before being summarized for all paticipants. The variables recorded related to sleep onset latency (SOL, the participant’s perceived ease of falling asleep (Likert scale)), total sleep time (TST) and sleep quality (how the participant felt when they woke up, refreshed, somewhat refreshed, fatigued). The results are shown in Tables 13:

TABLE 13

| Sleep Diary data | | Week 1 | Week 2 | Week 3 | Week 4 | Week 5 |
|---|---|---|---|---|---|---|
| Sleep onset latency (Last night I fell asleep: 1=easily, 2 = after some time, 3 = with difficulty) | | | | | | |
| | Number | 8 | 9 | 8 | 7 | 7 |
| Absolute values | Mean | 2.41 | 1.87 | 1.79 | 1.92 | 2.04 |
| | Std dev | 0.59 | 0.52 | 0.53 | 0.61 | 0.50 |
| | Median | 2.50 | 2.00 | 1.93 | 2.14 | 2.29 |
| | Minimum | 1.29 | 1.00 | 1.00 | 1.17 | 1.29 |
| | Maximum | 3.00 | 2.71 | 2.43 | 2.86 | 2.57 |
| Change from week 1 * | Mean | | -0.52 | -0.67 | -0.61 | -0.49 |
| | Std dev | | 0.50 | 0.41 | 0.68 | 0.24 |
| | Median | | -0.43 | -0.62 | -0.43 | -0.55 |
| | Minimum | | -1.50 | -1.55 | -1.67 | -0.71 |
| | Maximum | | 0.00 | -0.29 | 0.14 | 0.00 |
| T-test** | | p=0.021 | | p=0.007 | ns (0.088) | p=0.006 |
| Total sleep time (hours) | | | | | | |
| | Number | 8 | 8 | 7 | 6 | 6 |
| Absolute values | Mean | 6.53 | 6.84 | 7.86 | 7.62 | 7.71 |
| | Std dev | 1.86 | 1.24 | 1.63 | 1.89 | 1.71 |
| | Median | 6.66 | 6.43 | 7.00 | 7.00 | 7.56 |
| | Minimum | 3.86 | 5.21 | 5.93 | 5.20 | 5.29 |
| | Maximum | 9.17 | 8.80 | 9.79 | 10.24 | 10.36 |
| Change from week 1 * | Mean | | -0.01 | 1.05 | 1.21 | 1.29 |
| | Std dev | | 1.72 | 1.35 | 1.36 | 1.62 |
| | Median | | 0.36 | 0.80 | 0.93 | 1.32 |
| | Minimum | | -2.83 | -0.57 | -0.79 | -0.63 |
| | Maximum | | 2.59 | 3.50 | 3.29 | 3.47 |
| T-test** | | | ns | ns | ns | ns |
| Sleep quality (When I woke up for the day, I felt: 1 = refreshed, 2 = somewhat refreshed, 3 = fatigued) | | | | | | |
| | Number | 8 | 9 | 8 | 7 | 7 |
| Absolute values | Mean | 2.58 | 2.50 | 1.95 | 2.28 | 2.44 |
| | Std dev | 0.48 | 0.28 | 0.49 | 0.45 | 0.42 |
| | Median | 2.71 | 2.57 | 2.00 | 2.14 | 2.43 |
| | Minimum | 1.50 | 2.14 | 1.00 | 1.67 | 1.86 |
| | Maximum | 3.00 | 3.00 | 2.57 | 3.00 | 3.00 |
| Change from week 1* | Mean | | -0.04 | -0.60 | -0.41 | -0.25 |
| | Std dev | | 0.61 | 0.45 | 0.53 | 0.31 |
| | Median | | -0.10 | -0.54 | -0.43 | -0.14 |
| | Minimum | | -0.86 | -1.14 | -1.33 | -0.71 |
| | Maximum | | 1.07 | -0.14 | 0.29 | 0.14 |
| T-test** | | | ns | p=0.007 | ns (0.082) | ns |

* One Participant did not complete a week 1 diary. Changes for this participant for weeks 3, 4 and 5 were compared to week 2 data. **Paired t-test (comparison to week 1 sleep diary average data, two-tailed), ns = not significant at the 0.05 significance level; p=p-value.

The results show that there was an improvement in sleep latency for a total daily dose of 5 mg THC/5 mg CBD (stage 2), a total daily dose of 10 mg THC/10 mg CBD (stage 3); or a single dose of 12.5 mg THC/12.5 mg CBD (Stage 5), and an improvement in sleep quality at a total daily dose of 10 mg THC/10 mg CBD (stage 3), however, there was no improvement in the number of hours of sleep.

Pharmacokinetic studies were undertaken and the results are shown in Table 14:

TABLE 14

| | | 2.5 mg | 2.5 mg fed | 5 mg | 7.5 mg | 12.5 mg |
|---|---|---|---|---|---|---|
| THC | Cₘₐₓ (ng/mL) | 0.83 ± 0.29 (9) | 1.52 ± 0.55 (9) | 2.56 ± 1.70 (8) | 3.84 ± 1.62 (7) | 7.74 ± 5.14 (7) |
| | Tₘₐₓ (hr) | 1.67 ± 1.09 (9) | 2.78 ± 1.20 (9) | 2.06 ± 1.32 (8) | 1.36 ± 0.63 (7) | 2.14 ± 0.90 (7) |
| | AUC₀₋₈ (ng/mL*h) | 2.00 ± 1.38 (9) | 5.86 ± 2.07 (9) | 6.04 ± 2.07 (8) | 10.6 ± 3.84 (7) | 19.4 ± 9.62 (7) |
| | t_{½} (hr) | 2.60 ± 0.53 (4) | 2.63 ± 0.43 (4) | 2.47 ± 0.88 (6) | 2.41 ± 0.62 (7) | 3.40 ± 2.70 (6) |
| CBD | Cₘₐₓ(ng/mL) | 0.62 ±0.19 (9) | 0.99 ± 0.45 (9) | 1.51 ± 0.55 (8) | 2.26 ± 0.95 (7) | 4.53 ± 2.82 (7) |
| | Tₘₐₓ (hr) | 1.83 ± 1.00 (9) | 2.78 + 1.20 (9) | 1.75 ± 1.04 (8) | 1.64 ± 1.18 (7) | 2.29 + 1.25 (7) |
| | AUC₀₋₈ (ng/mL*h) | 1.76 ± 0.84 (9) | 3.67 + 1.57 (9) | 5.09 ± 1.80 (8) | 7.47 ± 2.95 (7) | 13.3 ± 6.67 (7) |
| | t_{½}(hr) | 4.25 ± 0.54 (4) | 2.95 ± 0.66 (4) | 4.54 ± 2.41 (7) | 3.47 ± 0.86 (6) | 3.56 ± 2.37 (6) |

On day 1, the participants received 2.5 mg of MC composition after a 12 hour fast, while on day 8, the participants received a high fat meal (about 60 g fat) 30 minutes before the 2.5 mg dose of MC. The presence of the high fat meal significantly increased Cₘₐₓ and AUC₀₋₈ for THC and CBD, with t_{½}being unchanged.

Generally, there was no indication that participant Body Mass Index (BMI) affected plasma concentration of THC or CBD, however, as the number of participants was small and all had a BMI > 25, it was not possible to draw a firm conclusion.

Generally, THC and CBD were detected in most time points that were sampled. The concentrations of THC were generally higher than the concentrations of CBD. There was a linear dose response relationship for both Cₘₐₓ and AUC₀₋₈ with increasing doses for THC and CBD. A high fat meal 30 minutes before dosing significantly increased both Cₘₐₓ and AUC₀₋₈ for THC and CBD. The high fat meal also generally delayed the Tₘₐₓ by at least 1 hour. The t_{½}did not change with increasing dose which was 2.5 hours for THC and 3.6 hours for CBD.

1. A pharmaceutical composition comprising a cannabinoid component and a terpene component, the composition comprising:
(a) a cannabinoid component comprising Δ⁹-tetrahydrocannabinol (THC), cannabidiol (CBD) wherein the cannabinoid component comprises:
i) ≥ 40% w/w Δ⁹-tetrahydrocannabinol (THC),
ii) ≤ 50% w/w cannabidiol (CBD),
iii) ≥ 0.15% w/w Cannabigerol (CBG),
iv) ≤ 0.5% w/w Cannabinodiol (CBN); and
(b) a terpene component comprising:
i) β-caryophyllene in an amount of ≥ 0.4% w/w of the composition,
ii) d-limonene in an amount of ≥ 0.2% w/w of the composition, and
iii) β-pinene in an amount of ≥ 0.15% w/w of the composition.

2. The pharmaceutical composition according to claim 1 wherein the ratio of THC:CBD is about 2:1 to about 1:1.

3. The pharmaceutical composition according to claim 2 wherein the ratio of THC:CBD/CBG is about 1:1.

4. The pharmaceutical composition according to claim 1 wherein the cannabinoid component is present in an amount of about 1% to about 10% w/w of the composition.

5. The pharmaceutical composition according to claim 4 wherein the cannabinoid component is present in an amount of about 2.5 to about 3.5% w/w of the composition.

6. The pharmaceutical composition according to claim 1 the terpene component is present in an amount of about 0.75% w/w to about 10% w/w of the composition.

7. The pharmaceutical composition according to claim 6 wherein the terpene component is present in an amount of about 0.75% w/w to about 1.25% w/w of the composition.

8. The pharmaceutical composition according to claim 1 wherein the β-caryophyllene is present in an amount of about 0.4% w/w to about 5% w/w of the composition.

9. The pharmaceutical composition according to claim 8 wherein the β-caryophyllene is present in an amount of about 0.4% w/w to about 0.5% w/w of the composition.

10. The pharmaceutical composition according to claim 1 wherein the d-limonene is present in an amount of about 0.2% w/w to about 5% w/w of the composition.

11. The pharmaceutical composition according to claim 10 wherein the d-limonene is present in an amount of about 0.2% w/w to about 0.3% w/w of the composition.

12. The pharmaceutical composition according to claim 1 wherein the β-pinene is present in an amount of about 0.15% w/w to about 5% w/w of the composition.

13. The pharmaceutical composition according to claim 12 wherein the β-pinene is present in an amount of about 0.15% w/w to about 0.25% w/w of the composition.

14. A method of treating chronic pain, comprising administering to a subject in need thereof an effective amount of the pharmaceutical composition of claim 1.

15. (canceled)

16. (canceled)

17. The method according to claim 14, wherein the subject is an athlete or retired athlete.

18. The method, according to claim 14 wherein the chronic pain is a result of injury sustained during training or sport.

19. The method, according to claim 14 wherein the chronic pain is a result of over use or repetitive action that develops over time.

20. The method, according to claim 14 wherein the chronic pain is arthritic pain, back pain, joint pain, inflammatory pain or nerve pain.
